(19) United States
(12) Patent Application Publication
(10) Pub. No.: US 2009/0285912 A1
(43) Pub. Date: Nov. 19, 2009

(54) Food Supplement and Method for Enhancing Athletic Performance

(76) Inventor: Ramiro Rodriquez, Dallas, TX (US)

Correspondence Address: LAWRENCE R. YOUST;Lawrence Youst PLLC, 2900 McKinnon, Suite 2208, DALLAS, TX, 75201, US

(21) Appl. No.: 12/122,299

(22) Filed: May 16, 2008

(51) Int. Cl. A61K 36/00

(52) U.S. Cl. 424/725

(57) ABSTRACT

Food supplements comprising Tribulus terrestris extract, folic acid and an orotic acid or a derivative thereof. The food supplements optionally, but preferably, include effective amounts of nutrients selected from the group consisting of Vitamin A, Vitamin E, Vitamin C, Vitamin B1, Vitamin B2, Vitamin B6, Vitamin B12 and combinations thereof. The food supplements are suitable for supplementing the diet of an athlete, for decreasing body fat and increasing strength, lean muscle mass and endurance.

### FIELD OF THE INVENTION

This invention relates, in general, to food supplements useful for decreasing body fat and increasing strength, lean muscle mass, and endurance in athletes, and, in particular, to food supplements comprising Tribulus terrestris extract, folic acid and an orotic acid or a derivative thereof and methods for supplementing the diet of an athlete using these supplements.

### BACKGROUND OF THE INVENTION

Athletes have a continuing need to enhance athletic performance by decreasing body fat and increasing strength, lean muscle mass, and endurance. Food supplements for achieving these purposes are growing in popularity among athletes. Synthetic hormone precursors have been shown to decrease body fat and increase strength and muscle mass, however, the use of synthetic hormone precursors can have significant negative side effects on an athlete's health. For instance, it has been found that regular use of anabolic steroids can result in elevated blood pressure, increased risk of heart disease, urinary and bowel problems, liver damage, cancer, severe acne, and premature baldness. In addition, other supplements that have been referred to in the market place as natural alternative to anabolic steroid, such as androstenedione and dehydroepiandrosterone, have been linked to side effects similar to those associated with anabolic steroids. Many of these supplements have been banned by collegiate and professional sports leagues and have been defined as controlled substances by the U.S. Congress.

Other common food supplements for increasing strength and muscle mass, such as creatine and its derivatives, are believed to result in fewer health side effects than synthetic hormone precursors. However, these supplements are not believed to effectively increase endurance or deliver effective amounts of nutrients to the diet of athletes. Athletes must therefore consume other supplements for these purposes.

Thus, there is a need among athletes for a new food supplement for decreasing body fat and increasing strength, lean muscle mass, and endurance while adding nutritional value to the diet of an athlete, and a method for supplementing the diet of an athlete using such a supplement.

### SUMMARY OF THE INVENTION

The present invention is a food supplement for enhancing athletic performance by decreasing body fat and increasing strength, lean muscle mass and endurance in athletes. The food supplement of the present invention decreases body fat, stimulate muscle growth and increases strength as an alternative to synthetic hormone precursors and creatine and its derivatives. The food supplement of the present invention contains a mineral carrier for efficiently delivering essential nutrients, which are provided in effective amounts, into muscle cells of an athlete.

The food supplements according to the present invention include Tribulus terrestris extract, folic acid and an orotic acid or a derivative thereof. The food supplements may optionally, and preferably, also include effective amounts of nutrients selected from the group consisting of Vitamin A, Vitamin E, Vitamin C, Vitamin B1, Vitamin B2, Vitamin B6, Vitamin B12 and combinations thereof. It has been determined that such food supplements may enhance athletic performance by decreasing body fat and increasing strength, lean muscle mass, and endurance in athletes.

### DETAILED DESCRIPTION OF THE INVENTION

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention, and they do not delimit the scope of the present invention.

The food supplements of the present invention comprise Tribulus terrestris extract, folic acid and an orotic acid or a derivative thereof.

Tribulus terrestris, also called puncturevine, is a medicinal herb native to the Mediterranean region which thrives in dry soil and warm climates. The preferred form of Tribulus terrestris in the present invention is that of an extract. Although the present invention is not intended to be limited by any theoretical explanation, Tribulus terrestris extract is believed to decrease body fat and increase strength and lean muscle mass by increasing levels of luteinizing hormone, which in turn stimulates the production of testosterone in males, which is known to promote the growth and strength of muscles and bones.

Orotic acid and its derivatives, such as its salts and esters, are thought to increase muscle strength by increasing carnosine levels and the formation of creatine phosphate in muscle cells. Orotates are mineral salts of orotic acid which are believed to act as carriers for transporting electrolytes and minerals throughout the body. Specifically, it is thought that binding orotic acid to a mineral salt enhances cellular uptake of the mineral atoms by allowing them to pass more easily through cell membranes because of the neutral charge of the orotate. Orotates include magnesium orotate, potassium orotate, lithium orotate, zinc orotate and calcium orotate.

Preferably, a salt of orotic acid is used in the food supplement according to the present invention. Potassium orotate is the most preferred orotic acid derivative. Again, without limiting by any theoretical explanation, potassium orotate is thought to facilitate tissue uptake of Vitamin C and increase blood levels of reduced gluthatione, a natural antioxidant.

In the food supplements according to the present invention, a preferred weight ratio of Tribulus terrestris extract to orotic acid or a derivative thereof is 1-1.5 to 3.0. More preferably, each food supplement comprises from about 50 mg to about 75 mg Tribulus terrestris extract, about 150 mg potassium orotate, and effective amounts of nutrients selected from the group consisting of Vitamin A, Vitamin E, Vitamin C, Vitamin B1, Vitamin B2, Vitamin B6, Vitamin B12, folic acid, and combinations thereof. In a preferred embodiment, the Vitamin B12 is in the form of methylcobalamin. In a preferred embodiment, the folic acid is in the form of L-methylfolate.

Most preferably, the food supplements of the present invention comprise Tribulus terrestris extract in an amount between about 50 mg and about 75 mg; potassium orotate in an amount of about 150 mg; Vitamin A in an amount between about 1,050 IU and about 1,300 IU; Vitamin E in an amount between about 50 mg and about 60 mg; Vitamin C in an amount between about 120 mg and about 160 mg; Vitamin B1 in an amount between about 2 mg and about 2.5 mg; Vitamin B2 in an amount between about 1 mg and about 1.5 mg; Vitamin B6 in an amount between about 2 mg and about 2.5 mg; Vitamin B12 in an amount between about 15 mcg and 20 mcg; and folic acid in an amount between about 900 mcg and about 990 mcg.

It is further preferred that the food supplement according to the present invention be in the form of a capsule.

It is a preferred embodiment of the methods according to the present invention to administer the food supplement 3 to 5 time during the day.

The following food supplement compositions and methods are given as illustrative examples and are not intended to limit the scope of the invention. In each example, the athlete is intended to consume 3-5 servings of the supplement as described herein daily.

### EXAMPLE 1

Each 318.015 mg capsule contains:

| | | |
|---|---|---|
| Tribulus Terrestris | 50.00 | mg |
| Potassium Orotate | 150.00 | mg |
| Vitamin A | 1,051 | IU |
| Vitamin B1 | 2.00 | mg |
| Vitamin B2 | 1.00 | mg |
| Vitamin B6 | 2.00 | mg |
| Vitamin B12 | 0.015 | mg |
| Vitamin C | 120.00 | mg |
| Vitamin E | 52.00 | mg |
| Folic Acid | 0.90 | mg |

### EXAMPLE 2

Each 318.105 mg capsule contains:

| | | |
|---|---|---|
| Tribulus Terrestris | 50.00 | mg |
| Potassium Orotate | 150.00 | mg |
| Vitamin A | 1,051 | IU |
| Vitamin B1 | 2.00 | mg |
| Vitamin B2 | 1.00 | mg |
| Vitamin B6 | 2.00 | mg |
| Vitamin B12 | 0.015 | mg |
| Vitamin C | 120.00 | mg |
| Vitamin E | 52.00 | mg |
| Folic Acid | 0.99 | mg |

### EXAMPLE 3

Each 444.915 mg capsule contains:

| | | |
|---|---|---|
| Tribulus Terrestris | 75.00 | mg |
| Potassium Orotate | 150.00 | mg |
| Vitamin A | 1,356 | IU |
| Vitamin B1 | 2.00 | mg |
| Vitamin B2 | 1.00 | mg |
| Vitamin B6 | 2.00 | mg |
| Vitamin B12 | 0.015 | mg |
| Vitamin C | 150.00 | mg |
| Vitamin E | 60.00 | mg |
| Folic Acid | 0.90 | mg |

### EXAMPLE 4

Each 458.220 mg capsule contains:

| | | |
|---|---|---|
| Tribulus Terrestris | 75.00 | mg |
| Potassium Orotate | 150.00 | mg |
| Vitamin A | 1,288 | IU |
| Vitamin B1 | 2.50 | mg |
| Vitamin B2 | 1.50 | mg |
| Vitamin B6 | 2.50 | mg |
| Vitamin B12 | 0.020 | mg |
| Vitamin C | 160.00 | mg |
| Vitamin E | 62.00 | mg |
| Folic Acid | 0.90 | mg |

1. A food supplement comprising:
Tribulus terrestris extract;
folic acid; and
an orotic acid or a derivative thereof.

2. The food supplement as recited in claim 1 wherein the orotic acid or a derivative thereof further comprises a salt or ester of orotic acid.

3. The food supplement as recited in claim 1 wherein the orotic acid or a derivative thereof further comprises potassium orotate.

4. The food supplement as recited in claim 3 further comprises a capsule containing the Tribulus terrestris extract in an amount between about 50 mg and about 75 mg and the potassium orotate in an amount of about 150 mg.

5. The food supplement as recited in claim 1 wherein the weight ratio of the Tribulus terrestris extract to the orotic acid or derivative thereof is about 1-1.5 to 3.0.

6. The food supplement as recited in claim 1 wherein the food supplement further comprises effective amounts of nutrients selected from the group consisting of Vitamin A, Vitamin E, Vitamin C, Vitamin B1, Vitamin B2, Vitamin B6, Vitamin B12 and combinations thereof.

7. The food supplement as recited in claim 6 further comprising a capsule containing:
Tribulus terrestris extract in an amount between about 50 mg and about 75 mg;
potassium orotate in an amount of about 150 mg;
Vitamin A in an amount between about 1,050 IU and about 1,300 IU;
Vitamin E in an amount between about 50 mg and about 60 mg;
Vitamin C in an amount between about 120 mg and about 160 mg;
Vitamin B1 in an amount between about 2 mg and about 2.5 mg;
Vitamin B2 in an amount between about 1 mg and about 1.5 mg;
Vitamin B6 in an amount between about 2 mg and about 2.5 mg;
Vitamin B12 in an amount between about 15 mcg and 20 mcg; and
folic acid in an amount between about 900 mcg and about 990 mcg.

8. The food supplement as recited in claim 1 wherein the food supplement is in the form of a capsule.

9. A method for supplementing the diet of an athlete comprising:
providing a food supplement comprising Tribulus terrestris extract, folic acid and an orotic acid or a derivative thereof; and
administering the food supplement to the diet of the athlete.

10. The method as recited in claim 9 wherein the orotic acid or a derivative thereof further comprises a salt or ester of orotic acid.

11. The method as recited in claim 9 wherein the weight ratio of the Tribulus terrestris extract to the orotic acid or derivative thereof is about 1-1.5 to 3.0.

12. The method as recited in claim 9 wherein from about 150 mg to about 375 mg Tribulus terrestris extract and from about 450 mg to about 600 mg orotic acid or a derivative thereof is administered to the diet of the athlete daily.

13. The method as recited in claim 9 wherein the orotic acid or a derivative thereof further comprises potassium orotate.

14. The method as recited in claim 13 wherein from about 150 mg to about 375 mg Tribulus terrestris extract, from about 450 mg to about 600 mg potassium orotate, from about 3,150 IU to about 6,500 IU Vitamin A, from about 150 mg to about 300 mg Vitamin E, from about 360 mg to about 800 mg Vitamin C, from about 6 mg to about 12.5 mg Vitamin B1, from about 3 mg to about 7.5 mg Vitamin B2, from about 6 mg to about 12.5 mg Vitamin B6, from about 45 mcg to about 100 mcg Vitamin B12, and from about 1.8 mg to about 4.95 mcg folic acid is administered to the diet of the athlete daily.

15. A food supplement consisting essentially of:
Tribulus terrestris extract, potassium orotate, Vitamin A, Vitamin E, Vitamin C, Vitamin B1, Vitamin B2, Vitamin B6, Vitamin B12 and folic acid.

16. The food supplement as recited in claim 15 further comprises the Tribulus terrestris extract in an amount between about 50 mg and about 75 mg and the potassium orotate in an amount of about 150 mg.

17. The food supplement as recited in claim 15 wherein the weight ratio of the Tribulus terrestris extract to the potassium orotate is about 1-1.5 to 3.0.

18. The food supplement as recited in claim 15 further comprising: Tribulus terrestris extract in an amount between about 50 mg and about 75 mg;
potassium orotate in an amount of about 150 mg;
Vitamin A in an amount between about 1,050 IU and about 1,300 IU;
Vitamin E in an amount between about 50 mg and about 60 mg;
Vitamin C in an amount between about 120 mg and about 160 mg;
Vitamin B1 in an amount between about 2 mg and about 2.5 mg;
Vitamin B2 in an amount between about 1 mg and about 1.5 mg;
Vitamin B6 in an amount between about 2 mg and about 2.5 mg;
Vitamin B12 in an amount between about 15 mcg and 20 mcg; and
folio acid in an amount between about 900 mcg and about 990 mcg.

19. The food supplement as recited in claim 15 wherein the food supplement is in the form of a capsule.
